(19) United States
(12) Patent Application Publication
(10) Pub. No.: US 2026/0202555 A1
(43) Pub. Date: Jul. 16, 2026

(54) System and Method for Monitoring Radiation Exposure in a Hospital Environment for an Operator

(71) Applicants: UBIQUICOM S.R.L., Milano (IT); FISMECO S.R.L., Roma (IT)

(72) Inventors: LUCA INDOVINA, ROMA (IT); STEFANO SARASSO, Milano (IT)

(73) Assignees: UBIQUICOM S.R.L., Milano (IT); FISMECO S.R.L., Roma (IT)

(21) Appl. No.: 19/362,935

(22) Filed: Oct. 20, 2025

Related U.S. Application Data
(63) Continuation of application No. 17/601,195, filed on Apr. 2, 2020, now abandoned.

(30) Foreign Application Priority Data
Apr. 5, 2019 (IT) 102019000005170

(51) Int. Cl. G01T 1/02; A61B 5/00; A61B 90/98

(52) U.S. Cl. CPC G01T 1/02; A61B 5/6801; A61B 90/98; A61B 2503/20

(57) ABSTRACT

A system for monitoring exposure to radiation in a hospital environment is disclosed where the system contains a dosimeter wearable by a health care worker, a radiation source identification means for identifying a source of radiation emitted by an X-ray apparatus, a position acquisition means for acquiring the position of the dosimeter, and a radiation exposure monitoring unit and a means for transmitting information to a remote server. A corresponding method and computer program for monitoring the exposure is further disclosed.

This application claims priority under 35 USC 120 as a continuation in part application of US Application No. 17/601,195 filed October 4, 2021, which is a national stage entry under 35 USC 371 of PCT/IB2020/053144, filed April 2, 2020, which in turn claims priority to Italian Application No. 102019000005170 filed April 5, 2019, the entire contents of all of which are incorporated by reference in their entireties.

### TECHNICAL FIELD

The present invention relates to a system and method for monitoring radiation exposure in a hospital environment for an operator.

In particular, the present invention relates to a method and system for monitoring the radiation exposure of an operator wearing a dosimeter and operating within a hospital area where ionizing radiation exposure is present.

### Prior Art

As is well known, the traditional dosimeters (both passive and active) currently used in hospitals and in particular in operating theatres (for example in Cardiology, Endoscopy, Therapy, General Surgery, Orthopaedics) present numerous problems that have not yet been solved, as they only allow measuring the ionizing radiation to which they are exposed.

Ionizing radiation comprises high energy electromagnetic waves (X-rays, gamma rays) and particles (α particles, β particles, neutrons). Ionizing radiation is emitted by radioactive elements and instruments such as X-ray apparatuses and radiation therapy equipment.

Most imaging methods using ionizing radiation (e.g., x-rays, CT, scintigraphy) expose patients to relatively low doses of radiation, generally considered harmless. However, any dose of ionizing radiation is potentially harmful, and there is no threshold below which no harmful effects occur, so every effort is made to minimize exposure to ionizing radiation.

To date, the radiation detection devices worn by an operator do not allow:

ensuring that they are actually worn by a specific operator (e.g. the personal dosimeter could simply be “placed” near the radiation source but not worn), and that, if worn, they are actually worn by the specific operator to which they are assigned, and positioned correctly (e.g. at chest height);

verifying that the prescribed Personal Protective Equipment (DPI) is also worn at the same time

knowing and monitoring the operator's actual distance from the radiation source, and therefore compliance with the prescribed minimum distances;

detecting and intervening in real time in the event of abnormal situations;

measuring how long the apparatuses are actually used;

measuring how much radiation is emitted during a specific procedure;

collecting detailed data on time periods that can be parametrized (Big Data) and related to all personnel involved, to allow Management to carry out precise and in-depth analyses and identify critical issues and related opportunities for improvement as well as for safety during exposure, including, for example, checks on the number of operators actually present during the service in the operating theatre, how long an average service lasts, how much an apparatus is actually used;

The known passive dosimeters allow data to be stored in the dosimeter itself (which is not connected) and communicated centrally only on a periodic basis (typically once a month), often manually, with the consequent possibility of making errors or omissions (e.g. to improperly benefit from the radiological risk allowances that in Italy legally envisage 15 days of holiday/year and a remuneration of 100 euros/month in relation to the activities carried out);

The known type of electronic dosimeters allow the operator to obtain an immediate reading of the radiation exposure, but the same is in no way centrally stored and its reading can however distract the operator during normal activity within the hospital area.

Only traditional dosimeters are used in Italy and abroad, which are not able to solve even one of the problems mentioned above.

One type of known dosimeter envisages electronic dosimeters connected to a traffic light in the operating theatre that indicates to the operator, during performance, the possible exceeding of a reference exposure level (red traffic light).

However, this solution does not allow for constant monitoring of the operator's position, working conditions, exposure time or whether Personal Protective Equipment (DPI) is worn correctly.

One drawback of the passive dosimeters and direct-read dosimeters is that they provide a simple exposure measurement that is not centrally stored and lacks any information on the place and mode of work in which the operator has been exposed to ionizing radiation (location, position with respect to the X-ray source, exposure time, DPI used).

### Object of The Invention

An object of the present invention is to detect and monitor in real time exposure to ionizing radiation (i.e. effective and/or equivalent dose) received by the operator during ionizing radiation activities (in particular, in operating rooms to ensure verification of the dose received during the activity) and the consequent failure to exceed the dose limits.

Another object of the present invention is to detect and monitor in real time the correct position (distance) of the operator with respect to the radiation source during performance, as optimizing the operator/source distance allows minimizing the doses for the operator himself as well as the exposure time in the individual activity.

A further object of the present invention is to detect and monitor in real time the presence of Personal Protective Equipment (DPI) worn by the operator to ensure the correct execution of risk containment procedures.

Another object of the present invention is to detect and monitor in real time the actual use of the dosimeter worn by the operator, and to also prevent fraudulent behaviour as much as possible.

A further object of the present invention is to detect and monitor in real time the verification of the operators actually present during operating theatre services and the amount of time in which the apparatuses are actually used.

Another object of the present invention is that of providing a method and system for monitoring exposure to radiation in a hospital environment that can guarantee a high level of safety.

A further object of the present invention is that of providing a method and system for monitoring exposure to radiation in a hospital environment that is in real time and efficient.

A further and not least object of the present invention is that of providing a method and system for monitoring exposure to radiation in a hospital environment that is highly reliable, easy to realize and simple to use.

In a first aspect of the invention, the above-mentioned objects are achieved by a system for monitoring exposure to radiation in a hospital environment according to that which is disclosed in claim 1.

Advantageous aspects are disclosed in dependent claims 2 to 13.

In a second aspect of the invention, the above-mentioned objects are achieved by an accommodation facility access method according to that which is disclosed in claim 14.

In a third aspect, the invention describes a computer program, which, when running on a computer, implements at least one or more steps of the method according to the second aspect of the invention, according to that which is disclosed in claim 15.

In general, the invention offers the following technical effects:

allows an increase in the safety of operators within hospital areas subject to ionizing radiation;

allows a reduction of the doses absorbed by the operators present within an area;

allows the personnel responsible for operator safety to implement procedures within the operating theatres for the various subjects/operators which optimise their interventions;

allows real-time monitoring of the risk level of each operator present in the operating theatre;

improves productivity and efficiency;

allows reducing the possibility of mistakes or fraud;

allows the real-time monitoring of the radiation emissions from medical apparatuses.

The technical effects/advantages mentioned, and other technical effects/advantages of the invention, will emerge in further detail from the description provided herein below of an example of embodiment provided by way of approximate and non-limiting example with reference to the appended drawings.

### BRIEF DESCRIPTION OF THE DRAWINGS

For a better understanding of the invention and to appreciate the advantages thereof, several non-limiting example embodiments are described herein below, referring to the attached figures, in which:

FIG. 1 is an overall view of the system of the invention; and

FIG. 2 is a block diagram of the system of FIG. 1.

### DETAILED DESCRIPTION OF PREFERRED EMBODIMENTS OF THE INVENTION

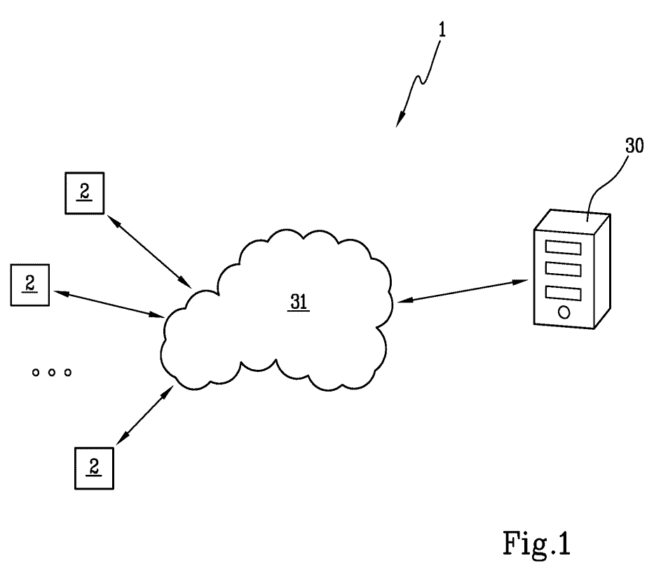

With reference to the cited figures, the system for monitoring exposure to radiation in a hospital environment according to the invention is indicated overall with the reference number 1 in the block diagram of FIG. 1.

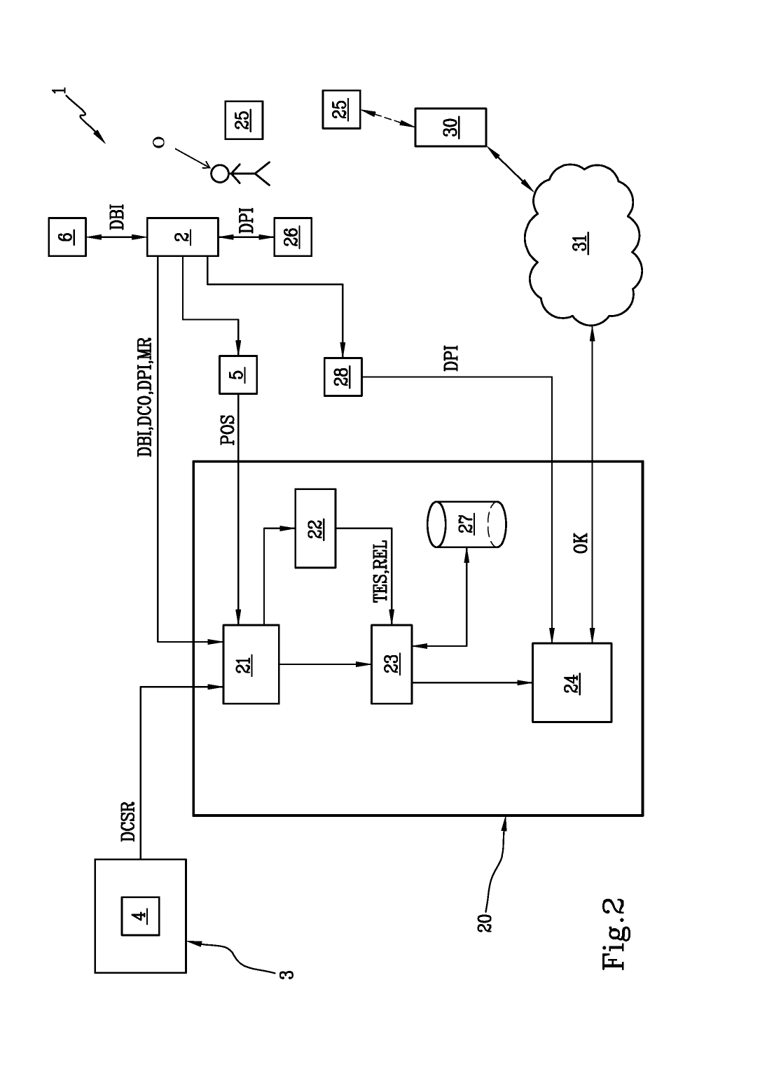

The system 1, shown in FIGS. 1 and 2, comprises a dosimeter 2 wearable by a health care worker O, configured to measure the radiation MR to which the worker O is exposed in the hospital environment, a radiation source identification means 4 for identifying a source of radiation emitted by a medical or X-ray apparatus 3, comprising emitted radiation source identification data DCSR, the procedure (PRC) in which the radiation is used and the position of the radiation source (POA) within an area of the hospital environment, a position acquisition means 5 for acquiring the position of the dosimeter 2, configured to communicate with said dosimeter 2 and to acquire the position in which it is located POS, a processing unit 20 configured to process data relating to the safety of each health care worker O and means for transmitting information and/or the conformity signal to a remote server 30.

According to an embodiment, the position acquisition 5 means comprises one or more antennas configured to send and receive radio frequency signals.

In a variation, the position acquisition means 5 employs Ultra Wide Band, UWB, technology for the tracking of the dosimeter 2, using techniques such as two way ranging, TWR, Phase Difference of Arrival, PDoA or Time Difference of Arrival, TDoA.

According to another embodiment, the position acquisition means 5 comprises one or more Bluetooth antennas employing a Bluetooth Low Energy, BLE, technology in particular in its Angle of Arrival implementation.

The present invention allows the medical apparatuses 3, radiation sources, and workers present in a hospital area to be managed safely and efficiently.

The invention further allows some hospital health safety procedures to be made more effective and simpler and assistance to be provided with the performance of the related procedures.

The dosimeter 2 is configured to measure the dose in terms of personal dose equivalent Hp(10) (radiation protection magnitude related to the overall health risk to the worker due to exposure to ionizing radiation). The effective dose is expressed in Sievert units (Sv) and submultiples thereof such as millisv (mSv). The dosimeter 2 could be configured to measure the dose at the ends, such as hands or crystalline, in terms of personal dose equivalent Hp(0.07), always in units of mSv. The magnitudes Hp(10) and Hp(0.07) allow an easy evaluation of the equivalent dose and effective dose radiation protection magnitudes with which they correlate. The equivalent dose is the absorbed dose, defined as the energy released by the radiation per mass unit, multiplied by a specific weighting factor for the type of radiation, which takes into account the effects on tissues based on the type of radiation delivered (e.g. X-rays, gamma rays, electrons). It is also expressed in sievert (Sv) or its submultiples such as millisievert (mSv). The effective dose is instead an estimate of the probabilistic risk of the worker sustaining damage over his lifetime in relation to the effective dose value attributed thereto; it takes into account the equivalent dose based on the sensitivity of the tissue exposed to radiation (e.g. the gonads are more sensitive). The effective dose is also expressed in Sv or its submultiples (mSv).

The dosimeter 2 contains therein a unique identification number (ID), stored in a read-only internal memory, the circuitry for the measurement of ionizing radiation and a means for communicating the measured radiation MR in short and medium range and the unique identification number ID.

Preferably, each dosimeter 2 is personal and is assigned to a specific health care worker O. When the dosimeter 2 is assigned to a health care worker, a pairing operation is performed, and the relative record is stored in a first memory unit 26 of the system. In this way, each record of the memory unit 26 will contain in a first field the unique identifier ID of the dosimeter, and in a second field the characteristic data of the health care worker DCO. In this way a logical association is made between personal dosimeter 2 and worker.

The processing unit 20 and the remote server 30 are provided with interfaces that allow them to communicate with each other through an electronic communication network 31.

FIG. 2 illustrates a non-limiting example of the invention, in which the processing unit 20 is arranged locally and the server 30 remotely. However, other non-limiting examples of the present invention contemplate the processing unit 20 and the server 30 arranged both locally or both remotely. Furthermore, in a further non-limiting embodiment of the present invention, the processing unit 20 and the server 30 coincide in a single device arranged locally or remotely.

The electronic network 31 is preferably the internet but could also be an intranet network or any private network adapted to implement a client-server type communication protocol. The electronic network 31 is connected, where necessary, to mobile networks for communication between the remote server 30 and the processing unit 20.

According to the invention, the system 1 comprises a processing unit 20.

The processing unit 20 comprises at least one input module 21 configured to receive from the dosimeter 2 the unique identification number (ID) and the measurement of ionizing radiation MR to which it is exposed while operating within an environment A in which a radiation source 3 is present.

According to a possible embodiment, the input module 21 may be implemented in the form of an application programming interface, API, in particular an API that follows the design principles of the REST architectural style.

The input module 21 receives from the radiation source identification means 4 characteristic data of the source of radiation emitted DCSR, data of the medical procedure delivered PRC in which it is used and the position POA of the medical or X-ray apparatus 3 (capable of emitting radiation) and receives from the position acquisition means 5, the position POS where the personal dosimeter 2 worn by the worker O is located.

A non-limiting example of a medical procedure delivered is coronary angioplasty, a radio-guided neurosurgery procedure to arrange a decomposed fracture of a limb.

The processing unit 20 further comprises a comparison module 23 configured to compare the data received by the input module 21 with corresponding predefined values stored in a second memory unit 27, such as for example average exposure time values for standard procedure, as well as average distances of a worker in said procedure or average effective dose and equivalent dose values in the procedures themselves.

The comparison module may be an electronic device or an electronic component comprised in a computer or an analogous device with computational capability that memorizes and run an algorithm configured to acquire as an input data the data of the dosimeter 2 from the input module 21 and then compare them with the predetermined values stored in the second memory unit 27.

Operatively, the comparison module 23 receives a numerical value from the dosimeter 2 quantifying the exposure of the user to radiations and compares said numerical value with a threshold fetched from the memory unit 27 and based on said comparison provides an output that allows to determine a risk level for the health of the user.

The processing unit 20 comprises a conformity module 24 configured to generate a conformity signal as a function of a correspondence OK that has occurred in the comparison performed by the comparison module 23.

Also, the conformity module may be an electronic device or an electronic component comprised in a computer or an analogous device with computational capability that memorizes and run an algorithm that in this case is configured to generate the conformity signal based on the comparison carried out by the comparison module 23.

By way of a non-limiting example the conformity module may be implemented as an application programming interface stored and run on a computer.

In general, it should be noted that in the present context and in the subsequent claims, the processing unit 20 is considered to be split into distinct functional modules (storage modules, processors, computers, electronic components and/or devices with computational capability) or operating modules) for the sole purpose of describing its functionalities clearly and completely.

Such processing unit can comprise a single electronic device, appropriately programmed to perform the functionalities described, and the different modules can correspond to hardware entities and/or routine software that are part of the programmed device.

Alternatively and/or additionally, these functionalities can be performed by a plurality of electronic devices on which the aforesaid functional modules can be distributed.

The processing unit 20 can also make use of one or more processors for executing the instructions contained in the memory modules.

The system 1 according to the invention advantageously comprises a display module 25 configured to generate a graphical representation or map of the radiation doses in the various positions within the hospital area by the operator O during an intervention or session in the presence of radiation sources.

The map can be made available and displayed to the worker O, for example on a personal electronic device (such as a tablet or smartphone) or on a screen in the area, or by another person located outside the area, for example on the remote server 30 or other remote personal device.

The dosimeter 2, the radiation source identification means 4 for identifying a source of radiation emitted by the medical or X-ray apparatus 3, the position acquisition means 5 of the dosimeter 2 are able to wirelessly communicate with the processing unit 20 through a plurality of wireless access points that are in electronic communication with the processing unit 20.

The plurality of wireless access points will be located within the area and will be configured not only to receive the wireless signals that are transmitted by the dosimeter 2, by the radiation source identification means 4, and by the position acquisition means 5, but also to communicate wireless signals to such devices.

In addition, the number of wireless access points is able to detect the specific location within area, for example, of the dosimeter 2 and/or medical or X-ray apparatus 3 at any time.

For example, the dosimeter 2 periodically measures, at predetermined time intervals, the dose of ionizing radiation per time unit within the area and communicates this accrual information to the processing unit 20, for example, communicates such accrual data to the wireless access points for communication to the processing unit 20.

At the same time, the wireless access points detect the position of the worker O (given by the position of the dosimeter 2 donned) along an x-axis, and a y-axis, and potentially along a z-axis, within the area, detecting the position of the dosimeter 2 and/or the medical or X-ray apparatus 3.

Advantageously, the position acquisition means 5 for acquiring the position of the dosimeter 2 comprises one or more between, at least, time difference of arrival and/or two way ranging and/or angle of arrival and/or distance measurement and/or RSSI measurement (Received Signal Strength Indicator), on technologies such as optical or acoustic (e.g. ultrasound) radio frequency.

Advantageously, the position of the worker O is given by a location tag arranged on the dosimeter 2 or on the personal identification badge of the worker O.

The information which can be stored (at regular or selected time intervals) in a memory unit comprises, for example, the amount of radiation dose absorbed, detected by the dosimeter 2, the position in which the dosimeter is located is at the time of measurement, the position of the radiation source 4, optionally, the radiation emitted by the medical or X-ray apparatus 3 at the same time, and further includes a time stamp reflecting the time when such measurement was made.

The positions occupied by a particular worker O within a certain area will be the points within the area where the various radiation dosage measurements were recorded.

Alternatively, the system records and displays on the map the relative distances between a given worker O and the radiation source 4 emitted. The measured doses can be displayed in numerical form on the map or by means of graphical representations such as different colours for the different intensities of absorbed radiation.

Dose intensity data can be taken directly from the measured dose intensity data set, or they can be normalised or otherwise processed.

The graphic representation thus obtained could be displayed to the operator himself, for his personal training, information and feedback, or it could be displayed or made available to a supervisor or technician or other individual interested in employee safety in such a way as to trace an exit and/or entry path from an area for the worker O along an optimized path to minimize radiation exposure.

In this way, the invention allows minimizing the dose absorbed by each operator present in the hospital area during a given treatment or operation.

The processing unit 20 comprises a calculation module 22 configured to calculate a radiation exposure time TES of the worker O and/or the relative distance REL between the position POS of the dosimeter 2 and the medical or X-ray apparatus 3, using the position data arriving at the position acquisition means 5.

The calculation module 22 may be an electronic device or an electronic component comprised in a computer or an analogous device with computational capability that memorizes and run an algorithm configured to fetch as an input the position data arriving at the position acquisition means 5 and use said information to calculate radiation exposure of the user (inputs are processed to calculate the information described above. For example: The dosimeter 2 could be configured to measure the dose at the ends, such as hands or crystalline, in terms of personal dose equivalent Hp(0.07), always in units of mSv) and the position of the user (the module calculates the position in terms of x, y, z on a map).Advantageously, the comparison module 23 is configured to compare the exposure time TES and/or said relative distance REL with predefined values stored in a second memory unit 27.

The dosimeter 2 wearable by the operator O according to the invention can advantageously comprise a biometric data DBI detector device 6 (e.g. fingerprints, colour and size of the iris, the retina, the shape of the hand, the palm of the hand, the shape of the ear, the physiognomy of the face, the tone of voice, etc.). In this case, the processing unit 20 is configured to make a comparison between at least one biometric data DBI of the health care worker O and the characteristic data of the worker DCO at the time the dosimeter 2 is donned. This coupling, biometric data detected DBI of a worker O and personal dosimeter 2, can be stored in a memory unit.

The biometric data detected DBI is advantageously sent to the input module 21 and the comparison module 24 will verify that the worker O is actually authorized to access the area during a given procedure PRC.

Advantageously, a module is present for detecting the personal protective equipment worn by the worker O. This detection module can be provided directly within the dosimeter 2 or outside it. An example of a detection technique is the ability of the detection module to read passive tags (e.g. RFID) applied to said DPI.

In this context, the detection module and overall, the presently disclosed system may comprise in at least some of its embodiment a tag for radio frequency identification, RFID; i.e. an active or a passive radio frequency tag and correspondingly an RFID antenna configured to read the signal of the RFID tag.

A further possible embodiment comprises a low energy consumption transmitter, such as a Bluetooth Low Energy beacon or a transmitter configured to use the Ant+ protocol.

Once whether or not the operator is wearing the assigned personal protection equipment DPI and/or the type of personal protection equipment DPI worn by the worker O is detected, this data is advantageously sent to the input module 21 and, subsequently, the comparison module 23 will verify that the operator O is wearing the personal protection equipment DPI assigned and necessary to carry out a certain procedure PRC within the area.

The characteristic data of the worker DCO can comprise one or more of, at least, personal data; identification data; biometric data; and/or health data. Preferably, the characteristic data of the emitted radiation source DCSR can comprise one or more between at least identification data; and/or ionizing radiation emission data.

The area of the hospital environment is a radiologically controlled area located within the health care environment.

Advantageously, the position acquisition means 5 is arranged to detect the position POS of at least one between the dosimeter 2, the badge of the worker O and/or the medical or X-ray apparatus 3 located within the area of the hospital environment.

Optionally, the health care worker's badge can contain a local electronic device containing its own characteristic data (DCO) and is capable of dialoguing with at least one between the dosimeter 2 and/or the position acquisition means 5 and/or the radiation exposure monitoring unit.

Preferably, the position acquisition means 5 is configured to store the position POS in association with the corresponding measured dose in a memory unit.

Preferably, placed in proximity to the zone of access to the area there is an identification device 28 configured to identify the dosimeter 2 or the worker O entering or exiting the hospital area or the personal identification badge.

Advantageously, the identification device 28 is in data connection with the processing unit 20, in particular, with the conformity module 24, and is able to send a significant signal DPI of whether or not the operator is wearing the required DPI.

If the worker O is not wearing the required personal protection equipment DPI, the conformity module 24 will generate a non-conformity signal KO, regardless of the outcome of the comparison carried out by the comparison module 23.

The identification means 4 of the medical or X-ray apparatus 3 consists of an RFID radio-frequency tag and/or an NFC tag and/or a barcode on various dimensions containing the characteristic data of the source of radiation emitted DCSR (for example, brand and model of the apparatus, date of the last revision, characteristic data of radiation emissions, etc.).

Optionally, an alarm module can be provided configured to send an alarm signal to the dosimeter 2 or to the electronic device given to the health care worker O as a function of a correspondence OK of the comparison made by said comparison module 23.

In a second aspect of the invention, a method for monitoring exposure to radiation in a hospital environment is provided comprising the steps of:

assigning a wearable dosimeter 2 to a health care worker O, the dosimeter 2 being configured to measure the radiation MR to which the worker (O) is exposed and comprising a unique identification number ID;

preparing on a medical or X-ray apparatus 3 a radiation source identification means 4 for identifying a source of radiation emitted by the apparatus 3 comprising data identifying the source of radiation emitted DCSR and the position of the apparatus 3 within an area of the hospital environment;

preparing a position acquisition means 5 for acquiring the position of the dosimeter 2, configured to communicate with said dosimeter 2 and to acquire the position in which it is located POS;

receiving the unique identification number ID and the measurement by said dosimeter 2 of the ionizing radiation MR to which he is exposed:

extracting from a first memory unit 26 characteristic data of the health care worker, using the unique identification number ID received from the dosimeter;

receiving said characteristic data of the source of radiation emitted DCSR by the medical of X-ray apparatus 3 from said means for identifying a source of radiation 4;

receiving the position POS in which the dosimeter 2 is located from said position acquisition means 5;

comparing the data received by the input module 21 with predefined values;

generating a conformity signal as a function of a correspondence OK that has occurred in the comparison performed;

transmitting the information and/or conformity signal to a remote server 30 via an information transmission means.

A third aspect of the present invention provides a computer program that, when running on a computer, implements at least one or more steps of the method according to the first aspect of the invention.

The method according to the invention further comprises the functional characteristics of the operating modules described in the system 1.

The above allows:

better safeguarding the safety of personnel through constant monitoring of radiation exposure and better scheduling of shifts, thus ensuring compliance with current legislation in terms of justification, optimisation and limitation of workers' exposure;

avoiding criminal sanctions, even up to the suspension or revocation of authorisations for the exercise of activities with ionizing radiation, imposed by the control bodies (in Italy ASL (Local Health Authority), Ministry, Provincial Labour Directorate) in the presence of the incorrect management of radiation protection safety for workers, especially in operating theatres, where the risk of exposure is considered very high;

avoiding those additional penalties typically paid when appropriate procedures involving the correct use of DPI are not documented.

As a person skilled in the art can easily understand, the invention allows overcoming the drawbacks highlighted above with reference to the prior art. In particular, the present invention allows improving the safety of the workers and means operating in the zone with exposure to ionizing radiation. It also enables better safety management by the supervisor.

It is clear that the specific characteristics are described in relation to different embodiments of the invention with an exemplary and non-limiting intent. Obviously, a person skilled in the art can make further modifications and variants to the present invention, in order to satisfy contingent and specific needs. For example, the technical characteristics described in relation to an embodiment of the invention can be extrapolated therefrom and applied to other embodiments of the invention. Such modifications and variations are moreover contained within the scope of the invention as defined by the following claims.

In general, it is noted that any of the embodiments described above can be implemented in association with computers such as laptops and desktop computers, or generally speaking, any processor/calculator with computational capability. The principles and advantages of the embodiments can be used for any system or apparatus, such as any processor, that could benefit from any of the embodiments described herein. The teachings herein are applicable to a variety of systems. Although this disclosure includes example embodiments, the teachings described herein can be applied to a variety of structures.

For example, the processors of the present invention may be used in conjunction with software, any requisite microchips, and the antennas as described herein to provide the processing capability to calculate a position of a user. The antennas as described herein may contain both receiving and transmitting abilities using RFID readers that employ the use of radio waves. The RFID tags may be passive tags or active tags and the active tags may have a power source that allows them to operate at long ranges.

Aspects of this disclosure can be implemented in various electronic devices. Examples of the electronic devices can include, but are not limited to, consumer electronic products, parts of the consumer electronic products such as wireless or cabled communication infrastructure, electronic test equipment, etc. Examples of the electronic devices can include, but are not limited to, wearable computing devices such as a smart watch or an ear piece, a computer monitor, a computer, a desktop computer, a laptop, a tablet, a hand-held computer, an Internet of things device, a system comprising one or more portable memory chips, a peripheral device, a wrist watch, etc.

We claim:

1. A system for monitoring exposure to radiation in a hospital environment, comprising:
a dosimeter wearable by a health care worker, configured to measure an ionizing radiation to which the worker is exposed and comprising an identification number
a radiation source identification means for identifying a source of radiation emitted by an X-ray apparatus, comprising radiation source identification data, a procedure wherein the X-ray apparatus is used and an X-ray apparatus position thereof within an area of the hospital environment;
a position acquisition device comprising at least a RFID and a processor, the position acquisition device configured to communicate with said dosimeter and to acquire a dosimeter position;
a radiation exposure monitoring unit comprising:
a first memory unit comprising characteristic data of the health care worker associated with said identification number of the dosimeter;
a processing unit configured to process data relating to a safety of each health care worker comprising:
an input module configured to receive:
from said dosimeter, the identification number and the measurement of ionizing radiation to which the healthcare worker is exposed;
from said radiation source identification means, said radiation source identification data, the procedure and the X-ray apparatus position;
wherein said identification means for identifying and X-ray apparatus consists of an RFID radio-frequency tag and/or and NFC tag containing said characteristic data of the source of radiation emitted;
from said position acquisition device, the dosimeter position;
a comparison module configured to compare the data received by the input module with predefined values;
a conformity module configured to generate a conformity signal as a function of a correspondence that has occurred in the comparison performed by said comparison module,
means for transmitting information to a remote server,
a detection module for detecting a personal protective equipment worn by the worker, configured to detect whether the worker is wearing one or more items of personal protective equipment.

2. The system according to claim 1, comprising a display module configured to generate a map of radiation doses absorbed by the worker in various positions within the area, and for each procedure.

3. The system according to claim 1, wherein the processing unit comprises a calculation module configured to calculate a time of exposure of the worker to the radiation and/or the relative distance between the position of the dosimeter and the X-ray apparatus and/or the measurement of the radiation received.

4. The system according to claim 3, wherein said comparison module is configured to compare said time of exposure and/or said relative distance with predefined values.

5. The system according to claim 1, wherein the dosimeter wearable by the worker is associated with a detection device for detecting biometric data.

6. The system according to claim 5, wherein said processing unit is configured to perform a comparison between at least one item of biometric data of the health care worker and the characteristic data of the worker at the moment when the dosimeter is donned.

7. The system according to claim 1, wherein the position acquisition device for acquiring the position of the dosimeter comprises one or more between, at least:
time difference of arrival;
two way ranging.

8. The system according to claim 1, wherein a position of the worker is given by a location tag positioned on the dosimeter or on a personal identification badge of the worker.

9. The system according to claim 1, wherein said characteristic data of the worker comprise one or more among, at least:
personal data;
identification data;
biometric data;
health data.

10. The system according to claim 1, wherein said characteristic data of the source of radiation emitted comprise one or more among, at least:
identification data;
data on emissions of ionizing radiation.

11. The system according to claim 1, wherein placed in proximity to the zone of access to the area there is an identification device configured to identify the dosimeter or the worker entering or exiting the hospital area or the personal identification badge.

12. A method for monitoring exposure to radiation in a hospital environment, comprising the steps of:
assigning a wearable dosimeter to a health care worker, the dosimeter being configured to measure the radiation to which the worker is exposed and associable with a characteristic data of the health care worker by means of a unique identification number;
preparing, on an X-ray apparatus a radiation source identification means for identifying a source of radiation emitted by the apparatus comprising data identifying the source of radiation emitted and an X-ray apparatus position (PA) within an area of the hospital environment, as well as a procedures in which the apparatus is used; wherein said identification means for identifying an X-ray apparatus consists of an RFID radio-frequency tag and/or an NFC tag containing said characteristic data of the source of radiation emitted;
preparing a position requisition device comprising a RFID and a processor configured to communicate with said dosimeter and to acquire a dosimeter position (POS);
receive said characteristic data of the health care worker and a measurement by said dosimeter of an ionizing radiation (MR) to which the healthcare worker is exposed;
receive said characteristic data of the source of radiation emitted by the X-ray apparatus from said means for identifying a source of radiation;
receive the dosimeter position from said position acquisition device;
compare the data received by an input module with predefined values;
generate a conformity signal as a function of a correspondence that has occurred in the comparison performed;
transmit an information to a remote server via an information transmission means;
preparing a detection module for detecting a personal protective equipment worn by the worker, configured to detect whether the worker is wearing one or more items of personal protective equipment.
